(19) United States
(12) Patent Application Publication
(10) Pub. No.: US 2006/0177849 A1
(43) Pub. Date: Aug. 10, 2006

(54) Nucleic acid primer set, nucleic acid probe set and method for detecting respiratory disease virus using the primer set and probe set

(76) Inventors: Ji-young Oh, Suwon-si (KR); Nam Huh, Seoul (KR); Jung-nam Lee, Incheon-si (KR)

Correspondence Address: CANTOR COLBURN, LLP, 55 GRIFFIN ROAD SOUTH, BLOOMFIELD, CT, 06002, US

(21) Appl. No.: 11/318,240

(22) Filed: Dec. 23, 2005

(30) Foreign Application Priority Data
Dec. 23, 2004 (KR) 10-2004-0112234

(51) Int. Cl. C12Q 1/68; C07H 21/04

(52) U.S. Cl. 435/6; 536/24.3

(57) ABSTRACT

Provided are a nucleic acid primer set capable of simultaneously amplifying the target sequences of five or more respiratory disease viruses and including oligonucleotides each of which comprises a fragment of at least 10 contiguous nucleotides present in sequences as set forth SEQ ID NOS: 1-26, a nucleic acid probe set for detecting the amplified product and a method of simultaneously detecting five or more respiratory disease viruses using the primer set.

### CROSS-REFERENCE TO RELATED PATENT APPLICATION

This application claims priority from Korean Patent Application No. 10-2004-0112234, filed on Dec. 24, 2004, in the Korean Intellectual Property Office, the disclosure of which is incorporated herein in its entirety by reference.

1. Field of the Invention

The present invention relates to a nucleic acid primer set capable of simultaneously amplifying the target sequences of five or more respiratory disease viruses, a probe set for detecting the amplified product and a method for detecting five or more respiratory disease viruses using the primer set and probe set.

2. Description of the Related Art

Respiratory infection is an important cause of death from infectious diseases. About 75% of acute respiratory diseases are caused by viruses. Most common viruses causing human respiratory diseases include measles virus, enterovirus, rhinovirus, SARS-associated coronavirus (SARS-coV), varicella zoster virus (VSV), adenovirus, human parainfluenza virus 1 (HPIV 1), human parainfluenza virus 2 (HPIV 2), human parainfluenza virus 3 (HPIV 3), influenza virus A (IVA), influenza virus B (IVB), respiratory syncytial virus A (RSVA), and respiratory syncytial virus B (RSVB). Rapid and specific detection of these viruses is essential for diagnosis, prevention, and treatment of respiratory diseases.

U.S. Pat. No. 5,744,299 discloses a method for detecting HPIV 1 by RT-PCR using primers capable of specifically amplifying a HPIV 1 sequence. U.S. Pat. Laid-Open Publication No. 2003/0130497 discloses a method for simultaneously detecting seven respiratory disease-associated viruses, i.e., HPIV 1, 2, 3, IVA, IVB, RSV, and adenovirus.

However, there are still no reports of a method of simultaneously detecting up to 13 respiratory disease viruses including measles virus, enterovirus, rhinovirus, SARS-coV, and VSV.

### SUMMARY OF THE INVENTION

The present invention provides a primer set capable of simultaneously amplifying five or more respiratory disease viruses.

The present invention also provides a method for simultaneously detecting five or more respiratory disease viruses using the primer set.

The present invention also provides a probe set for detecting at least one virus among 13 viruses.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates agarose gel electrophoretic results for PCR products after multiplex PCR using primers of SEQ ID NOS: 1 through 26; and

FIG. 2 illustrates hybridization results of target amplification products with probes of SEQ ID NOS: 27 through 50 immobilized on a microarray.

### DETAILED DESCRIPTION OF THE INVENTION

The present invention provides a nucleic acid primer set comprising at least one oligonucleotide of 10 to 100 nucleotides in length, selected from the group consisting of oligonucleotides each of which comprises a fragment of at least 10 contiguous nucleotides present in a sequence as set forth in SEQ ID NO: 1 and at least one oligonucleotides of 10 to 100 nucleotides in length, selected from the group consisting of oligonucleotides each of which comprises a fragment of at least 10 contiguous nucleotides present in a sequence as set forth in SEQ ID NO: 2; at least one oligonucleotide of 10 to 100 nucleotides in length, selected from the group consisting of oligonucleotides each of which comprises a fragment of at least 10 contiguous nucleotides present in a sequence as set forth in SEQ ID NO: 3 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 4; at least one oligonucleotide of 10 to 100 nucleotides in length, selected from the group consisting of oligonucleotides each of which comprises a fragment of at least 10 contiguous nucleotides present in a sequence as set forth in SEQ ID NO: 5 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 6; at least one oligonucleotide of 10 to 100 nucleotides in length, selected from the group consisting of oligonucleotides each of which comprises a fragment of at least 10 contiguous nucleotides present in a sequence as set forth in SEQ ID NO: 7 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 8; and at least one oligonucleotide of 10 to 100 nucleotides in length, selected from the group consisting of oligonucleotides each of which comprises a fragment of at least 10 contiguous nucleotides present in a sequence as set forth in SEQ ID NO: 9 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 10.

The nucleic acid primer set of the present invention may further include at least one oligonucleotide of 10 to 100 nucleotides in length, selected from the group consisting of oligonucleotides each of which comprises a fragment of at least 10 contiguous nucleotides present in a sequence as set forth in SEQ ID NO: 11 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 12; at least one oligonucleotide of 10 to 100 nucleotides in length, selected from the group consisting of oligonucleotides each of which comprises a fragment of at least 10 contiguous nucleotides present in a sequence as set forth in SEQ ID NO: 13 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 14; at least one oligonucleotide of 10 to 100 nucleotides in length, selected from the group consisting of oligonucleotides each of which comprises a fragment of at least 10 contiguous nucleotides present in a sequence as set forth in SEQ ID NO: 15 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 16; at least one oligonucleotide of 10 to 100 nucleotides in length, selected from the group consisting of oligonucleotides each of which comprises a fragment of at least 10 contiguous nucleotides present in a sequence as set forth in SEQ ID NO: 17 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 18; at least one oligonucleotide of 10 to 100 nucleotides in length, selected from the group consisting of oligonucleotides each of which comprises a fragment of at least 10 contiguous nucleotides present in a sequence as set forth in SEQ ID NO: 19 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 20; at least one oligonucleotide of 10 to 100 nucleotides in length, selected from the group consisting of oligonucleotides each of which comprises a fragment of at least 10 contiguous nucleotides present in a sequence as set forth in SEQ ID NO: 21 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 22; at least one oligonucleotide of 10 to 100 nucleotides in length, selected from the group consisting of oligonucleotides each of which comprises a fragment of at least 10 contiguous nucleotides present in a sequence as set forth in SEQ ID NO: 23 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 24 or at least one oligonucleotide of 10 to 100 nucleotides in length, selected from the group consisting of oligonucleotides each of which comprises a fragment of at least 10 contiguous nucleotides present in a sequence as set forth in SEQ ID NO: 25 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 26; and combinations of the foregoing oligonucleotides.

The nucleic acid primer set of the present invention includes oligonucleotide pairs capable of amplifying five or more respiratory disease viruses, preferably up to 13 respiratory disease viruses. The nucleic acid primer set of the present invention can be used for amplifying a plurality of target sequences in one amplification reaction. The amplification reaction may be multiplex PCR but is not limited thereto.

Primer pairs contained in the nucleic acid primer set of the present invention have the following characteristics and can be used in simultaneous detection of five or more respiratory disease-associated viruses.

(1) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 1 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 2 are a primer pair specific for measles virus, and are selected from a conserved region of the F gene of 61 strains of measles virus.

(2) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 3 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 4 are a primer pair specific for enterovirus, and are selected from a conserved region within the 5′ UTR of 3 strains of enterovirus.

(3) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 5 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 6 are a primer pair specific for rhinovirus, and are selected from a conserved region within the 5′ UTR (polyprotein gene) of 6 strains of rhinovirus.

(4) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 7 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 8 are a primer pair specific for SARS-associated coronavirus (SARS-coV), and are selected from a conserved region of the GD69 gene of 75 strains of SARS-coV.

(5) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 9 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 10 are a primer pair specific for varicella zoster virus (VSV), and are selected from a conserved region of the ORF54 gene of 7 strains of VSV.

(6) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 11 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 12 are a primer pair specific for adenovirus, and are selected from a conserved region of the Hexon gene of 31 strains of adenovirus.

(7) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 13 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 14 are a primer pair specific for human parainfluenza virus 1 (HPIV 1), and are selected from a conserved region of the HN gene of 43 strains of HPIV 1.

(8) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 15 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 16 are a primer pair specific for human parainfluenza virus 2 (HPIV 2), and are selected from a conserved region of the HN gene of 6 strains of HPIV 2.

(9) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 17 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 18 are a primer pair specific for human parainfluenza virus 3 (HPIV 3), and are selected from a conserved region of the HN gene of 14 strains of HPIV 3.

(10) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 19 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 20 are a primer pair specific for influenza virus A (IVA), and are selected from a conserved region of the MP gene of 186 strains of IVA.

(11) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 21 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 22 are a primer pair specific for influenza virus B (IVB), and are selected from a conserved region of the NP gene of 35 strains of IVB.

(12) The at least one oligonucleotide of 10 to 100 nucleotides in length, selected from the group consisting of oligonucleotides each of which comprises a fragment of at least 10 contiguous nucleotides present in a sequence as set forth in SEQ ID NO: 23 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 24; and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 25 and the at least one oligonucleotide of 10 to 100 nucleotides in length, selected from the group consisting of oligonucleotides each of which comprises a fragment of at least 10 contiguous nucleotides present in a sequence as set forth in SEQ ID NO: 26 are primer pairs specific for respiratory syncytial virus A (RSVA) and respiratory syncytial virus B (RSVB), and are selected from a conserved region of the F gene of 28 strains of RSVA and RSVB.

The virus-specific primer pairs of the present invention are selected by analyzing viral sequences available from a public database (e.g., Genbank) using a public program (e.g., DNASTAR Inc., Madison, Wis. 53715, America). First, conserved regions of various viral strains are identified, and primer pairs are selected from these conserved regions. Each primer pair can be tested in PCR using a template target sequence derived from a standard viral sample to ensure that a specific amplification product is produced. When a primer pair is combined with other primer pairs for PCR, i.e., when multiplex PCR is performed, PCR products corresponding to the primer pairs can be obtained. This shows that the primer pairs contained in the primer set of the present invention can implement chain extension through polymerization without affecting PCR reaction by intramolecular or intermolecular interaction. PCR products obtained by PCR using the primer pairs of the present invention are of different lengths, and thus can be easily specifically identified by electrophoresis, etc.

The present invention also provides a method for simultaneously detecting multiple respiratory disease viruses, the method including:

obtaining a nucleic acid from a sample;

amplifying the nucleic acid using the nucleic acid primer set of the present invention; and

detecting an amplification product,

wherein when an amplification product specific for a target virus is detected, it is determined that the target virus is present.

In the present invention, the operation of obtaining the nucleic acid from the sample may be performed by a nucleic acid extraction method known in the art. For example, a phenolchloroform extraction method or a purification method using a nucleic acid-binding solid material (e.g., U.S. Pat. No. 5,234,809) may be used.

In an embodiment of the present invention, the operation of obtaining the nucleic acid from the sample may include separating RNA from the sample and obtaining cDNA from RNA. For example, the sub-operation of obtaining cDNA may be performed using reverse transcriptase. Reverse transcription using reverse transcriptase may be coupled with PCR (called “RT-PCR”).

In the method of the present invention, the operation of amplifying the nucleic acid may be performed by PCR.

In the method of the present invention, the operation of detecting the amplification product may include hybridizing the amplification product with a probe set. The probe set may include at least one oligonucleotide of 10 to 100 nucleotides in length, selected from the group consisting of oligonucleotides comprising a fragment of at least 10 contiguous nucleotides present in sequences as set forth in SEQ ID NOS: 27-50 and their complementary sequences.

In the method of the present invention, the probe sequences of 10 to 100 nucleotides in length, selected from the group consisting of oligonucleotides each of which comprises a fragment of at least 10 contiguous nucleotides present in sequences of SEQ ID NOS: 27-50 and their complementary sequences are specifically bound to PCR products, amplified by PCR using the nucleic acid primer set of the present invention, specific for measles virus, enterovirus, rhinovirus, SARS-coV, VSV, adenovirus, HPIV 1, HPIV 2, HPIV 3, IVA, IVB, RSVA and RSVB. The probe sequences of the present invention are selected from conserved regions of target sequences to be amplified by the nucleic acid primer set of the present invention.

In the method of the present invention, the probe set may include at least one oligonucleotide of 10 to 100 nucleotides in length, selected from the group consisting of oligonucleotides each of which comprises a fragment of at least 10 contiguous nucleotides present in a sequence of SEQ ID NO: 27 or 28 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 29 or 30 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 31 or 32 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 33 or 34 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 35 or 36 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 37 or 38 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 39 or 40 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 41 or 42 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 43 or 44 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 45 or 46 and their complementary sequences; at least one oligonucleotide of 10 to 100 nucleotides in length, selected from the group consisting of oligonucleotides each of which comprises a fragment of at least 10 contiguous nucleotides present in a sequence of SEQ ID NO: 47 or 48 and their complementary sequences; and at least one oligonucleotide of 10 to 100 nucleotides in length, selected from the group consisting of oligonucleotides each of which comprises a fragment of at least 10 contiguous nucleotides present in a sequence of SEQ ID NO: 49 or 50 and their complementary sequences.

In the present invention, the probe set may be immobilized on a solid support such as a film, a glass, or a plastic polymer. Immobilization of probes on a surface of a solid support may be performed by a probe immobilization method known in the art. For example, the immobilization of probes on a surface of a solid support may be performed by baking at 80° C. or UV curing. In addition, synthesis of a probe polymer on an activated solid substrate by photolithography (WO 92/100092), covalent attachment of previously synthesized probes onto an activated substrate surface (spotting), etc. may be used. In an embodiment of the method of the present invention, the probe set may be immobilized on a microarray substrate.

In the present invention, a PCR product may be labeled for easy detection. A labeling material for nucleic acids is well known in the art. For example, a fluorescence material such as Cy3 and Cy5 may be used. The labeling material is not particularly limited provided that it can emit a detectable signal. For example, there may be used an enzyme capable of producing a radioactive material or a colormetric material.

The present invention also provides a probe oligonucleotide set for detecting at least one virus selected from the goup consisting of measles virus, enterovirus, rhinovirus, SARS-associated coronavirus, varicella zoster virus, adenovirus, human parainfluenza viruses 1, 2, and 3, influenza viruses A and B, and respiratory syncytial viruses A and B, comprising at least one oligonucleotide of 10 to 100 nucleotides in length selected from the group consisting of oligonucleotides each of which comprises a fragment of at least 10 contiguous nucleotides present in sequences as set forth in SEQ ID NOS: 27-50 and their complementary sequences.

In the probe oligonucleotide set of the present invention, the probe set may include at least one oligonucleotide of 10 to 100 nucleotides in length, selected from the group consisting of oligonucleotides each of which comprises a fragment of at least 10 contiguous nucleotides present in a sequence as set forth in SEQ ID NO: 27 or 28 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 29 or 30 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 31 or 32 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 33 or 34 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 35 or 36 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 37 or 38 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 39 or 40 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 41 or 42 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 43 or 44 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 45 or 46 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 47 or 48 and their complementary sequences; and at least one oligonucleotide of 10 to 100 nucleotides in length, selected from the group consisting of oligonucleotides each of which comprises a fragment of at least 10 contiguous nucleotides present in a sequence as set forth in SEQ ID NO: 49 or 50 and their complementary sequences.

In the probe oligonucleotide set of the present invention, the probe set may include the oligonucleotides as set forth in SEQ ID NOS: 27-50.

The present invention also provides a microarray having the probe oligonucleotide set of the present invention immobilized thereon.

The probe oligonucleotide set and the microarray of the present invention are as described hereinbefore in the method of the present invention.

Hereinafter, the present invention will be described more specifically with reference to the following examples. The following examples are for illustrative purposes and are not intended to limit the scope of the invention.

### EXAMPLE 1

### Selection of Primers for Amplification of Target Sequences Specific for 13 Respiratory Disease Viruses

In this Example, a primer set capable of detecting target sequences specific for 13 viruses, i.e., measles virus, enterovirus, rhinovirus, SARS-coV, VSV, adenovirus, HPIV 1, HPIV 2, HPIV 3, IVA, IVB, RSVA, and RSVB was designed.

First, respiratory disease viral sequences were available from the Genbank and conserved regions were selected form the viral sequences using the DNASTAR program. The number of sequences used for analysis (the number of strains), sequence identification numbers, and the preserved regions are listed in Table 1 below.

TABLE 1
Sequences used for analysis and preserved regions

| | Conserved | |
|---|---|---|
| Virus | region | |
| species | or gene | The number of strains: used sequences |
| Measles | L gene | 61: NC_001405, NC_001460, AY271307, |
| virus | | NC_004001, NC_002067, NC_003266, |
| | | AF065066, AY128640, AB052911, X74662, |
| | | AJ012091, AB053166, AY008279, AF515814, |
| | | U20821, X76549, AF542129, AF542104, |
| | | AF542120, AF542128, AY224392, AF542122, |
| | | NC_001454, X51782, AB018424, AB162772, |
| | | AB098564, AB018427, AB067668, X98360, |
| | | AB018425 etc. |
| Entero- | 5′-UTR | 3; ETU22521, CXA9CG, AF251359 |
| virus | (polyprotein) | |
| Rhino- | 5′-UTR | 6; PIHRV14, HRV, HRV89, HRVACG, HRVPP, |
| virus | (polyprotein) | PIHRV2G |
| SRS | GD69 | 75; NC_004718, AY323977, AY485278, |
| CoV | | AY394998, AY350750, AY282752, AY485277, |
| | | AY310120, AY345987, AY345986, AY278491, |
| | | AY394990, AY394989, AY345988, AY394991, |
| | | AY394993, AY394992, AY291451, AY502928, |
| | | AY502925, AY357075, AY278554, AY502929, |
| | | AY502930, AY502927, AY502931, AY502926, |
| | | AY362699, AY502923, AY278487, AY362698, |
| | | AP006557, AY502932, AY278741, AY321118, |
| | | AP006560, AP006559, AY291315, AY357076, |
| | | AP006558, AY278490, AY279354, AY508724, |
| | | AY502924, AY394850, AY313906, AY278488, |
| | | AY427439, AY283794, AY304495, AY283796, |
| | | AY283798, AY394987, AY283797, AY283795, |
| | | AY394983, AY461660, AY297028, AY338174, |
| | | AY348314, AY338175, AY463059, AY395000, |
| | | AY395001, AY395002, AY394999, AY394985, |
| | | AY304488, AY278489, AY394995, AY394986, |
| | | AY390556, AY394996, AY394997 etc. |
| VZV | ORF54 | 7; X04370, AB097932, AB097933, AF206304, |
| | | AY379115, AY379116 etc. |
| Adeno- | Hexon | 31; NC_001405, NC_001460, AY271307, |
| virus | | NC_004001, NC_002067, NC_003266, |
| | | AF065066, AY128640, AB052911, X74662, |
| | | AJ012091, AB053166, AY008279, AF515814, |
| | | U20821, X76549, AF542129, AF542104, |
| | | AF542120, AF542128, AY224392, AF542122, |
| | | NC_001454, X51782, AB018424, AB162772, |
| | | AB098564, AB018427, AB067668, X98360, |
| | | AB018425 etc. |
| HPIV1 | HN | 43; NC_003461, M86781, M86783, M86780, |
| | | M86782, AF016280, M86786, M86785, M86789, |
| | | M86787, M86790, M86788, M86791, M86784, |
| | | M31228, U70943, U70938, U70941, U70936, |
| | | U70937, U70947, U70942, U70940, M91648, |
| | | U70948, U70945, U70939, U70944, U70946, |
| | | X55803, U01075, U01074, U01073, U01076, |
| | | U01081, U01079, U01082, U01080, U01077, |
| | | U01085, U01083, U01078, U01084 etc. |
| HPIV2 | HN | 14; NC_003443, AF533011, D00865, |
| | | AF533012, AF213352, AF533010 |
| HPIV3 | HN | 6; NC_001796, Z11575, M18759, M18763, |
| | | L25350, Z26523, U51116, M18764, M18762, |
| | | AY283063, M20402, M17641, M21649, M18760 |
| IVA | MP | 186; AF398867, AF258516, D00603, M59334, |
| | | M59330, AJ628066, M59331, M59329, D00601, |
| | | D00599, M59333, M59332, M63751, M63752, |
| | | M76604, M63749, M23976, M81577, M81571, |
| | | M81583, M63750, AY210068, AY210066, |
| | | AY210070, AY210067, M63753, AY210074, |
| | | AY210069, AJ238021, U02086, AY210071, |
| | | X15890, AY210083, AY210081, AY210080, |
| | | AY210079, AY210078, AY210077, AY210075, |
| | | J02137, AY210076, AY210073, AY210088, |
| | | AY210086, AJ628067, AY210103, AY210087, |
| | | AY210085, AY210082, AY210072, AY210089, |
| | | D00051, M14922, AF348180, AY210093, |
| | | AY210090, AY210222, AY210216, AY210215, |
| | | AY210214, L07346, L07345, L07340, |
| | | AY210092, AY210091, AY210220, AY210219, |
| | | AY210218, AY210217, AY210213, AY210212, |
| | | AY210211, AF348183, AF348182, AF348181, |
| | | AY210104, AY210101, AY210095, AY210084, |
| | | AY210223, AY210210, L07347, AY210100, |
| | | AY210094, AY210228, AY210225, AY210209, |
| | | AY210208, AY210102, AY210230, AY210224, |
| | | AY210221, AY210229, AY210096, AY210207, |
| | | AY210099, AY210097, AY210236, AF258517, |
| | | AF389119, V01084, M38279, Z54292, Z54290, |
| | | Z54291, M30746, J02147, M63755, M76602, |
| | | M76610, L24394, M76606, AF342819, M63754, |
| | | M22577, L07365, L07367, L07360, L07370, |
| | | L07353, L07364, L07352, L07369, L07372, |
| | | L07373, L07366, L07357, L07374, L07354, |
| | | L07359, L07362, L07363, L07361, D00600, |
| | | L07371, L07368, D00602, X51972, X52262, |
| | | AB019358, AJ458277, L07351, L07350, L07358, |
| | | L07356, AB019361, AF038254, L07355, |
| | | M76605, AF072545, L07349, U71145, |
| | | AF038257, AF038255, U71147, U71146, |
| | | L07343, AF115285, AF084276, AF255747, |
| | | AF255757, AF255745, AF046092, AF255765, |
| | | AF255753, AF255751, AF255750, AF036359, |
| | | AF028710, AF084278, AF255755, AJ291401, |
| | | AF084277, AF255746, AJ291400, AF255759, |
| | | AJ289873, AF115284, AF255744, AF255752, |
| | | AJ289871, AF255742, AF255743, AJ289872, |
| | | AF255761, AF255767 |
| IVB | NP | 35: M20174, AF100360, K01395, AF100359, |
| | | AF100357, AF100358, X14217, K01139, |
| | | M20173, L49385, L49384, AF100369, |
| | | AF100363, AF100368, AF100367, AF100366, |
| | | AF100362, AF100361, AF100371, AF100364, |
| | | AF100365, AF100372, AY044169, AF005739, |
| | | AF100370, AY260953, AY260946, AB036876, |
| | | AF170569, AB036874, AB036875, AB059255, |
| | | AF100373, AB059247 |
| RSVA | F gene | 28: U39661, U39662, M74568, AY114151, |
| | | AY114150, D00151, U31561, U31560, Z26524, |
| | | AY114149, L25351, U31558, U31562, X02221, |
| | | AY330615, AF512538, U31559, AY198176, |
| | | M11486, AY198175, AY330616, M22643, |
| | | AY330614, AY330612, AY330611, AY198177, |
| | | AF067125, D00334, AF013254 |
| RSVB | F gene | 28: U39661, U39662, M74568, AY114151, |
| | | AY114150, D00151, U31561, U31560, Z26524, |
| | | AY114149, L25351, U31558, U31562, X02221, |
| | | AY330615, AF512538, U31559, AY198176, |
| | | M11486, AY198175, AY330616, M22643, |
| | | AY330614, AY330612, AY330611, AY198177, |
| | | AF067125, D00334, AF013254 |

Primers of SEQ ID NOS: 1-26 specific for measles virus, enterovirus, rhinovirus, SARS CoV, VZV, adenovirus, HPIV1, HPIV2, HPIV3, IVA, IVB, RSVA and RSVB were selected from these preserved regions.

### EXAMPLE 2

### Detection of 13 Respiratory Disease-Associated Viruses on Agarose Gel

In this Example, 13 respiratory disease-associated viruses were cultured, viral genomes were separated from the cultures, multiple RT-PCR for the viral genomes were performed using the primers of SEQ ID NOS: 1-26, and PCR products were identified on agarose gel.

(1) Preparation of Viral Culture

With respect to viral DNA genomes, 5.5 μl of 5 N NaOH was added to a 50 μl culture solution until the final concentration reached 0.5 N. Then, the viral DNA genomes were denatured at 37° C. for 15 minutes and neutralized with 5.5 μl of 5 N HCl followed by dilution with H₂O. Viral RNA genomes were isolated from 200 μl of a cell supernatant using about 600 μl of a TriZol-reagent. The final precipitate obtained by precipitation with ethanol was stored at −20° C. after being resuspended in DEPC distilled water or was ready to used in RT-PCR.

(2) Reverse Transcription and PCR

cDNAs were synthesized using the 13 template nucleic acids derived from the viral cultures and hexamers or oligo-dT primers. That is, cDNAs were prepared using 0.5 μl of 5× Buffer 3, 0.5 μl of RNase inhibitor, 3 μl of dNTPs, random hexamer, 0.5 μl of superscript II RTase (reverse transcriptase), and 0.5 , μl of RNAs, treated with DEPC distilled water until the final volume reached 15 μl , incubated at 37° C. for 40 minutes and then at 42° C. for 90 minutes, and then incubated at 95° C. for 5 minutes to inactivate the enzyme. PCR was performed in a PCR mixture including 10 μl of cDNA, 4 μl of dNTPs, 1 μl (20 pmol/μl ) of each of the primers of SEQ ID NOS: 1-26, and 0.5 μl of Taq polymerase, according to the following thermal cycles: 30 cycles at 94° C. for 30 seconds, at 55° C. for 30 seconds, and at 70° C. for 30 seconds to thereby amplify target sequences specific for the viral genomes.

(3) Detection of Amplification Products

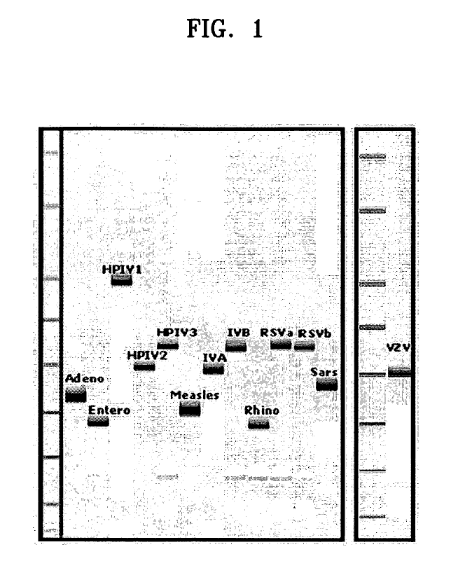

PCR products were analyzed by electrophoresis on an agarose gel. FIG. 1 illustrates agarose gel electrophoretic results for PCR products after multiplex PCR using the primers of SEQ ID NOS: 1-26. As shown in FIG. 1, 13 target sequences were identified. This shows that the specificity of each of the primers of SEQ ID NOS: 1-26 is not affected by the presence of the other primers in multiplex PCR. The lengths of the PCR products of FIG. 1 are presented in Table 2 below.

TABLE 2

| Virus | Length (bp) |
|---|---|
| Adenovirus | 182 |
| Enterovirus | 158 |
| HPIV1 | 346 |
| HPIV2 | 231 |
| HPIV3 | 255 |
| IVA | 151 |
| IVB | 260 |
| Measles virus | 174 |
| Rhinovirus | 158 |
| RSVA | 256 |
| RSVB | 256 |
| SARS-coV | 208 |
| VZV | 217 |

### EXAMPLE 3

### Detection of 13 Respiratory Disease-Associated Viruses on Microarray

In this Example, the PCR products obtained in Example 2 were hybridized with probes immobilized on a microarray, and the degree of hybridization was evaluated to detect the presence of a PCR product specific for a target virus.

Sample preparation and RT-PCR were performed in the same manner as in Example 2 except that a primer tagged with 5′-Cy3-labeled oligonucleotide of SEQ ID NO: 51 was used for a forward primer and a primer tagged with 5′-Cy3-labeled oligonucleotide of SEQ ID NO: 52 was used for a reverse primer. The detection of PCR products on the microarray was performed as follows.

(1) Probe Design

Probes were selected from amplification regions of the viral genomes using the DNAstar program, and the probe sequences are presented in Table 3 below.

TABLE 3

| Target virus | Probe sequence (SEQ ID NO:) |
|---|---|
| Measles virus | 27 |
| | 28 |
| Enterovirus | 29 |
| | 30 |
| Rhinovirus | 31 |
| | 32 |
| SARS coV | 33 |
| | 34 |
| VZV | 35 |
| | 36 |
| Adenovirus | 37 |
| | 38 |
| HPIV1 | 39 |
| | 40 |
| HPIV2 | 41 |
| | 42 |
| HPIV3 | 43 |
| | 44 |
| IVA | 45 |
| | 46 |
| IVB | 47 |
| | 48 |
| RSVA and RSVB | 49 |
| | 50 |

(2) Manufacturing of Probe-Immobilized Microarray

A microarray used in this Example was manufactured by adding the probes of SEQ ID NOS: 27-50 on a substrate activated with an amine compound followed by incubation at 50° C. for 30 minutes. Samples containing PCR products amplified from the 13 viral genomes were added to spots of the microarray on which virus-specific oligonucleotides were immobilized, and then incubated at 16° C. for 12 hours for hybridization.

(3) Detection

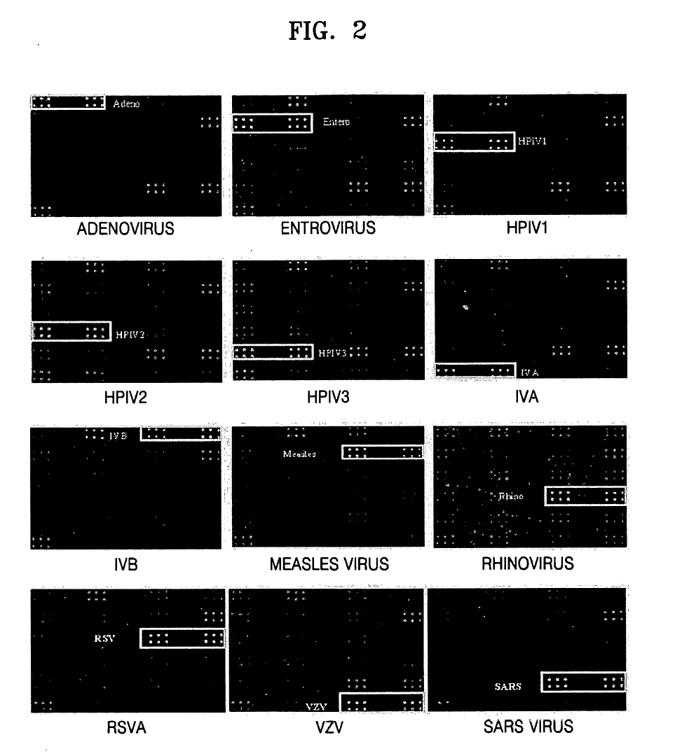

After the hybridization, the microarray was washed with a washing buffer and fluorescence was measured at 540 nm. FIG. 2 illustrates hybridization results of target amplification products with probes of SEQ ID NOS: 27 through 50 immobilized on a microarray.

According to a nucleic acid primer set of the present invention, five or more respiratory disease viruses can be simultaneously amplified.

According to a detection method of the present invention, five or more respiratory disease viruses can be simultaneously amplified with high specificity.

1. A nucleic acid primer set comprising at least one primer set selected from the group consisting of primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 1 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 2; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 3 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 4; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 5 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 6; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 7 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 8; and primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 9 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 10.

2. The nucleic acid primer set of claim 1, further comprising at least one primer set selected from the group consisting of primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 11 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 12; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 13 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 14; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 15 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 16; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 17 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 18; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 19 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 20; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 21 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 22; primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 23 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 24,(?? ??) or primer sets of at least one oligonucleotide of 10 to 100 nucleotides in length, selected from the group consisting of oligonucleotides each of which comprises a fragment of at least 10 contiguous nucleotides present in a sequence as set forth in SEQ ID NO: 25 and at least one oligonucleotide of 10 to 100 nucleotides in length, selected from the group consisting of oligonucleotides each of which comprises a fragment of at least 10 contiguous nucleotides present in a sequence as set forth in SEQ ID NO: 26.

3. A method for detecting a respiratory disease virus, the method comprising:
obtaining a nucleic acid from a sample;
amplifying the nucleic acid using the nucleic acid primer set of claim 1; and
detecting an amplification product,
wherein when an amplification product specific for a target virus is detected, it is determined that the target virus is present.

4. The method of claim 3, wherein the operation of obtaining the nucleic acid from the sample comprises:
isolating RNA from the sample; and
obtaining cDNA from the isolated RNA.

5. The method of claim 3, wherein the operation of amplifying the nucleic acid is performed by PCR.

6. The method of claim 3, wherein the operation of detecting the amplification product comprises:
hybridizing the amplification product with a probe set; and
detecting the degree of the hybridization, and
wherein the probe set comprises at least one oligonucleotide of 10 to 100 nucleotides in length, selected from the group consisting of oligonucleotides each of which comprises a fragment of at least 10 contiguous nucleotides present in sequences as set forth in SEQ ID NOS: 27-50 and their complementary sequences.

7. The method of claim 6, wherein the probe set comprises at least one oligonucleotide of 10 to 100 nucleotides in length, selected from the group consisting of oligonucleotides each of which comprises a fragment of at least 10 contiguous nucleotides present in a sequence as set forth in SEQ ID NO: 27 or 28 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 29 or 30 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 31 or 32 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 33 or 34 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 35 or 36 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 37 or 38 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 39 or 40 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 41 or 42 and their complementary sequences; at least one oligollucleotide of 10 to 100 nucleotides in length, selected from the group consisting of oligonucleotides each of which comprises a fragment of at least 10 contiguous nucleotides present in a sequence as set forth in SEQ ID NO: 43 or 44 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 45 or 46 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 47 or 48 and their complementary sequences; and at least one oligonucleotide of 10 to 100 mucleotides in length, selected from the group consisting of oligonucleotides each of which comprises a fragment of at least 10 contiguous nucleotides present in a sequence as set forth in SEQ ID NO: 49 or 50 and their complementary sequences.

8. The method of claim 6, wherein the probe set comprises the oligonucleotides as set forth in SEQ ID NOS: 27-50.

9. The method of claim 7, wherein the probe set is immobilized on a microarray substrate.

10. A probe oligonucleotide set for detecting at least one virus selected from the goup consisting of measles virus, enterovirus, rhinovirus, SARS-associated coronavirus, varicella zoster virus, adenovirus, human parainfluenza viruses 1, 2, and 3, influenza viruses A and B, and respiratory syncytial viruses A and B, comprising at least one oligonucleotide of 10 to 100 nucleotides in length selected from the group consisting of oligonucleotides each of which comprises a fragment of at least 10 contiguous nucleotides present in sequences as set forth in SEQ ID NOS: 27-50 and their complementary sequences.

11. The probe oligonucleotide set of claim 10, comprising at least one oligonucleotide of 10 to 100 nucleotides in length, selected from the group consisting of oligonucleotides each of which comprises a fragment of at least 10 contiguous nucleotides present in a sequence as set forth in SEQ ID NO: 27 or 28 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 29 or 30 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 31 or 32 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 33 or 34 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 35 or 36 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 37 or 38 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 39 or 40 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 41 or 42 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 43 or 44 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 45 or 46 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 47 or 48 and their complementary sequences; and at least one oligonucleotide of 10 to 100 nucleotides in length, selected from the group consisting of oligonucleotides each of which comprises a fragment of at least 10 contiguous nucleotides present in a sequence as set forth in SEQ ID NO: 49 or 50 and their complementary sequences.

12. The probe oligonucleotide set of claim 10, comprising the oligonucleotides as set forth in SEQ ID NOS: 27-50.

13. A microarray having the probe oligonucleotide set of any one of claim 10 immobilized thereon.

14. A method for detecting a respiratory disease virus, the method comprising:
obtaining a nucleic acid from a sample;
amplifying the nucleic acid using the nucleic acid primer set of claim 2; and
detecting an amplification product,
wherein when an amplification product specific for a target virus is detected, it is determined that the target virus is present.

15. The method of claim 14, wherein the operation of obtaining the nucleic acid from the sample comprises:
isolating RNA from the sample; and
obtaining cDNA from the isolated RNA.

16. The method of claim 14, wherein the operation of amplifying the nucleic acid is performed by PCR.

17. The method of claim 14, wherein the operation of detecting the amplification product comprises:
hybridizing the amplification product with a probe set; and
detecting the degree of the hybridization, and
wherein the probe set comprises at least one oligonucleotide of 10 to 100 nucleotides in length, selected from the group consisting of oligonucleotides each of which comprises a fragment of at least 10 contiguous nucleotides present in sequences as set forth in SEQ ID NOS: 27-50 and their complementary sequences.

18. The method of claim 17, wherein the probe set comprises at least one oligonucleotide of 10 to 100 nucleotides in length, selected from the group consisting of oligonucleotides each of which comprises a fragment of at least 10 contiguous nucleotides present in a sequence as set forth in SEQ ID NO: 27 or 28 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 29 or 30 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 31 or 32 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 33 or 34 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 35 or 36 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 37 or 38 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 39 or 40 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 41 or 42 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 43 or 44 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 45 or 46 and their complementary sequences; at least one oligonucleotide of 10 to 100 nucleotides in length, selected from the group consisting of oligonucleotides each of which comprises a fragment of at least 10 contiguous nucleotides present in a sequence as set forth in SEQ ID NO: 47 or 48 and their complementary sequences; and at least one oligonucleotide of 10 to 100 nucleotides in length, selected from the group consisting of oligonucleotides each of which comprises a fragment of at least 10 contiguous nucleotides present in a sequence as set forth in SEQ ID NO: 49 or 50 and their complementary sequences.

19. The method of claim 17, wherein the probe set comprises the oligonucleotides as set forth in SEQ ID NOS: 27-50.

20. The method of claim 8, wherein the probe set is immobilized on a microarray substrate.

21. The method of claim 18, wherein the probe set is immobilized on a microarray substrate.

22. The method of claim 19, wherein the probe set is immobilized on a microarray substrate.


SEQUENCE LISTING (52 sequences)

SEQ ID NO: 1 - DNA, 20 bp, Artificial sequence
  Measles virus L gene specific primer
ccagacackgcagctgattc

SEQ ID NO: 2 - DNA, 19 bp, Artificial sequence
  Measles virus L gene specific primer
tgaggaayaggattgcyga

SEQ ID NO: 3 - DNA, 19 bp, Artificial sequence
  Enterovirus 5′ UTR region specific primer
ccctgaatgyggctaacct

SEQ ID NO: 4 - DNA, 20 bp, Artificial sequence
  Enterovirus 5′ UTR specific primer
cractactttgggtgtccgt

SEQ ID NO: 5 - DNA, 19 bp, Artificial sequence
  Rhinovirus 5′ UTR region specific primer
ccctgaatgyggctaacct

SEQ ID NO: 6 - DNA, 20 bp, Artificial sequence
  Rhinovirus 5′ UTR region specific primer
cractactttgggtgtccgt

SEQ ID NO: 7 - DNA, 22 bp, Artificial sequence
  SARS virus GD69 specific primer
ttctcatggtggacagcttttg

SEQ ID NO: 8 - DNA, 23 bp, Artificial sequence
  SARS virus GD69 specific primer
ggaggaagacaaatcctatccag

SEQ ID NO: 9 - DNA, 20 bp, Artificial sequence
  VZV ORF54 specific primer
catatacgtatttaatggtg

SEQ ID NO: 10 - DNA, 20 bp, Artificial sequence
  VZV ORF54 specific primer
gttattccacgcagaaactc

SEQ ID NO: 11 - DNA, 19 bp, Artificial sequence
  Adenovirus specific primer
tggcgcathccvttctcca

SEQ ID NO: 12 - DNA, 18 bp, Artificial sequence
  Adenovirus specific primer
atcccatggaygagccca

SEQ ID NO: 13 - DNA, 20 bp, Artificial sequence
  HPIV1 HN gene specific primer
cctatgacatcaacgacaac

SEQ ID NO: 14 - DNA, 19 bp, Artificial sequence
  HPIV HN gene specific primer
ggtggcwtaacaactccgc

SEQ ID NO: 15 - DNA, 20 bp, Artificial sequence
  HPIV2 HN gene specific primer
agacycattggtgttacact

SEQ ID NO: 16 - DNA, 20 bp, Artificial sequence
  HPIV2 HN gene specific primer
gcaaaagctgttcagtcact

SEQ ID NO: 17 - DNA, 20 bp, Artificial sequence
  HPIV3 HN gene specific primer
aattactgggggtcagaagg

SEQ ID NO: 18 - DNA, 20 bp, Artificial sequence
  HPIV3 HN gene specific primer
tggggacattcatgyccara

SEQ ID NO: 19 - DNA, 20 bp, Artificial sequence
  Influenza virus A MP specific primer
tamccgaggtcgaaacgtac

SEQ ID NO: 20 - DNA, 20 bp, Artificial sequence
  Influenza virus A MP specific primer
ggctaaagacaagaccratc

SEQ ID NO: 21 - DNA, 20 bp, Artificial sequence
  Influenza virus B NP gene specific primer
accccracagagataaaraa

SEQ ID NO: 22 - DNA, 20 bp, Artificial sequence
  Influenza virus B NP specific primer
gaatgccagagaygtcaaag

SEQ ID NO: 23 - DNA, 20 bp, Artificial sequence
  RSV A and B F gene specific primer
trtcattratcaatgatatg

SEQ ID NO: 24 - DNA, 19 bp, Artificial sequence
  RSV A and B  F gene specific primer
cctctatgyacmaccaaca

SEQ ID NO: 25 - DNA, 20 bp, Artificial sequence
  RSV A and B  F gene specific primer
tgtcattaatcaatgatatg

SEQ ID NO: 26 - DNA, 19 bp, Artificial sequence
  RSV A and B F gene specific primer
tgttggttgtacatagagg

SEQ ID NO: 27 - DNA, 23 bp, Artificial sequence
  Measles virus specific probe
aartacacccarcaaagaagggt

SEQ ID NO: 28 - DNA, 21 bp, Artificial sequence
  measles virus specific probe
atggytggatgtrgtgaggaa

SEQ ID NO: 29 - DNA, 22 bp, Artificial sequence
  Enterovirus specific probe
tcgtaagggcaactctgcagcg

SEQ ID NO: 30 - DNA, 21 bp, Artificial sequence
  Enterovirus specific probe
ggttccgctgcagagttgccc

SEQ ID NO: 31 - DNA, 24 bp, Artificial sequence
  Rhinovirus specific probe
gtcgtaatgagcaattccgggacg

SEQ ID NO: 32 - DNA, 23 bp, Artificial sequence
  Rhinovirus specific probe
tcccggaattgctcattacgacc

SEQ ID NO: 33 - DNA, 27 bp, Artificial sequence
  SARS virus specific probe
acaaatgtaaatgcatcatcatcggaa

SEQ ID NO: 34 - DNA, 27 bp, Artificial sequence
  SARS virus specific probe
tgatggctataccatgcatgctaacta

SEQ ID NO: 35 - DNA, 27 bp, Artificial sequence
  VZV specific probe
acccttccatttaaaccactggtatag

SEQ ID NO: 36 - DNA, 26 bp, Artificial sequence
  VZV specific probe
aaaggtacgatcagaacagaatggaa

SEQ ID NO: 37 - DNA, 23 bp, Artificial sequence
  Adenovirus specific probe
gcgctrgayatgacttttgaggt

SEQ ID NO: 38 - DNA, 26 bp, Artificial sequence
  Adenovirus specific probe
rgayatgacttttgaggtggatccca

SEQ ID NO: 39 - DNA, 27 bp, Artificial sequence
  HPIV 1 speecific probe
atgttctgtamtagstgcaggaacaag

SEQ ID NO: 40 - DNA, 27 bp, Artificial sequence
  HPIV 1 specific probe
ttgttcctgcasctaktacagaacatg

SEQ ID NO: 41 - DNA, 24 bp, Artificial sequence
  HPIV 2 specific probe
atctaaccagtatttagcaatggg

SEQ ID NO: 42 - DNA, 22 bp, Artificial sequence
  HPIV 2 specific probe
taaatggttttcatagtcctga

SEQ ID NO: 43 - DNA, 25 bp, Artificial sequence
  HPIV 3 specific probe
tggcataaygtgytatcaagaccag

SEQ ID NO: 44 - DNA, 26 bp, Artificial sequence
  HPIV 3 specific probe
gtcttgataccacyttatgccatgtc

SEQ ID NO: 45 - DNA, 23 bp, Artificial sequence
  Influenza virus A specific probe
ggctaaagacaagaccratcctg

SEQ ID NO: 46 - DNA, 23 bp, Artificial sequence
  Influenza virus specific probe
agagwacgtacgtttcgacctcg

SEQ ID NO: 47 - DNA, 25 bp, Artificial sequence
  Influenza virus B specific probe
aaccagatgatggtcaaagctggrc

SEQ ID NO: 48 - DNA, 27 bp, Artificial sequence
  Influenza virus B specific probe
ctgcaacbgatgacaagaaaactgaat

SEQ ID NO: 49 - DNA, 24 bp, Artificial sequence
  RSV A specific probe
ccagcaaagttaytctatcatgtc

SEQ ID NO: 50 - DNA, 25 bp, Artificial sequence
  RSV A specific probe
ttyttctgrtcatttgttataggca

SEQ ID NO: 51 - DNA, 26 bp, Artificial sequence
  5′ Cy3 labelled tagging sequence for the forward primer
ataaagctagagactggccatctcaa

SEQ ID NO: 52 - DNA, 26 bp, Artificial sequence
  5′ Cy3 labelled tagging sequence for the reverse primer
gacaggaacttggagagaattaggta